(19) United States
(12) Patent Application Publication
(10) Pub. No.: US 2025/0135044 A1
(43) Pub. Date: May 1, 2025

(54) MULTIVALENT GLYCO-COMPLEX, IMAGING AGENT AND USES THEREOF

(71) Applicant: National Atomic Research Institute, Taoyuan (TW)

(72) Inventors: HUNG-WEN YU, Taoyuan (TW); Kun-Liang Lin, Taoyuan (TW); Mei-Hui Wang, Taoyuan (TW)

(21) Appl. No.: 18/384,436

(22) Filed: Oct. 27, 2023

(51) Int. Cl. A61K 51/04

(52) U.S. Cl. CPC A61K 51/0491; A61K 51/0474; A61K 2123/00

(57) ABSTRACT

The present disclosure relates to a multivalent glyco-complex, an imaging agent and uses thereof. The multivalent glyco-complex includes a plurality of glucose molecules, each of which connects to a central nitrogen atom through a linker, and a chelating group G. The multivalent glyco-complex can be used as an imaging agent to diagnose cancers and to evaluate the therapeutic efficacy of cancers.

### TECHNICAL FIELD

The present disclosure relates to the field of medical imaging, in particular to the field of medical imaging using a multivalent glyco-complex as shown in Formula (10).

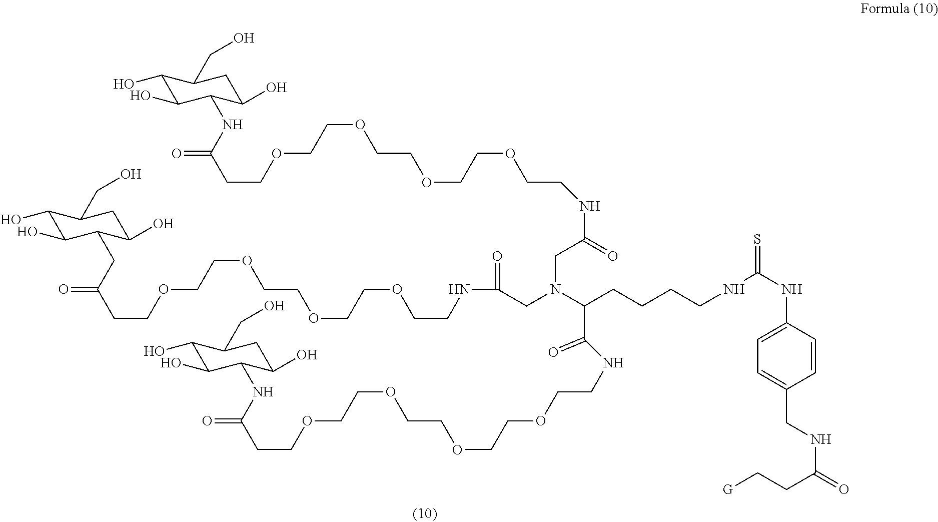

### BACKGROUND

Malignant tumors are a major public health issue worldwide, which are also one of the leading causes of death in the United States. The American Cancer Society pointed out in a report published on Jan. 12, 2023, that although the cancer mortality rate in the United States has shown a steadily decline over the past 30 years, new cases of breast cancer, uterine cancer, and prostate cancer are continually increasing. The probability of American men being diagnosed with any invasive cancer in their lifetime is about 40.9%, and for women, it's about 39.1%. The report also estimated that in 2023, there may be nearly 2 million new cancer cases in the United States, which, in other words, is equivalent to about 5,000 cases per day, and more than 600,000 people may die from cancer.

The latest (2020) cancer registry report released by National Health Administration, Ministry of Health and Welfare, Taiwan in 2023 stated that the number of new cancer cases was 121,979, an increase of 725 cases compared to 2019. The number of cancer deaths in 2020 was 50,161, accounting for 29.0% of total deaths, with a death rate of 212.7 people per 100,000 population, and the standardized death rate was 117.3 people per 100,000 population. Malignant tumors have been the leading cause of death in the country for 41 consecutive years, and the “cancer clock” continues to speed up, 3 seconds faster than in 2021, with an average of 1 person diagnosed with cancer every 4 minutes and 19 seconds. The above data clearly indicate that the number of cancer patients and the size of the related medical market are substantial. Even more, the number of people suffering from cancer continues to increase.

Early diagnosis and treatment are the key to the successful treatment of malignant tumors. If malignant tumors can be detected in the early stage and followed by appropriate treatment given to the patient, many of the cancer patients may have the opportunity to significantly improve their survival rates. For example, if the patients with colorectal cancer, breast cancer, etc., are given appropriate treatments in the early stages of cancer, they are observed having better prognosis.

Most of malignant tumors were known having the characteristic of high glucose usage rate, in view of this, the glucose analogs, such as ¹⁸F-FDG (2-Deoxy-2-fluoro-D-glucose), are often used clinically for cancer diagnosis. However, the use of ¹⁸F-FDG often encounters limitations and restrictions. For example, the preparation of ¹⁸F-FDG is not only comprising extremely cumbersome processes, it also requires the use of a cyclotron, an expensive equipment which is not equipped in most of the domestic medical hospitals, to produce F-18. Further, the preparation process of ¹⁸F-FDG requires the use of a synthesis cassette, and also requires additional steps such as dehydration, fluorination and deprotection to obtain ¹⁸F-FDG, which leads longer synthesis time.

Further, because tissues or organs metabolizing glucose in the body also takes up ¹⁸F-FDG, thus, when using ¹⁸F-FDG as a contrast agent for diagnosis cancers, extremely high background value will be appeared and observed in patient's brain and heart. This makes it difficult to distinguish between normal and tumor tissues in these organs and their surrounding areas using the imaging results of ¹⁸F-FDG. It is therefore limited in the use of cancer detections. Besides, it is also observed higher ¹⁸F-FDG up-taking in inflamed tissue, making the use of ¹⁸F-FDG contrast agent difficult to differentiate between tumor(s) and inflamed tissue(s) as well. As can be seen, ¹⁸F-FDG is not only having complicated and time-consuming preparation processes, but also possesses low specificity in the use of cancer diagnosis. In view of the aforementioned, there is an urgent need of multivalent glyco-complex in the art, with better imaging discrimination to improve the insufficiency of the prior art.

### SUMMARY

To provide a reader with a basic understanding of the present disclosure, the summary provides a brief description of the disclosure. The summary is not a complete description of the disclosure, and is not intended to limit the technical features or the scope of this application.

In order to discover a cancer diagnostic drug with a better discrimination and specificity, especially an imaging agent used in the cancer diagnosis, the present disclosure demonstrates and provides a multivalent glyco-complex, having a representative molecular structure as Formula (10), which contains three glucose molecules connecting individually to a central nitrogen atom with a linker, and a chelating group G; wherein the linker contains at least a polyethylene glycol (PEG) molecule, and the chelating group G connects to the linker at one end closed to the central nitrogen atom in the form of p-NCS-benzyl-(chelating group G). The chelating group G is selected from a group consisting of: 1,4,7-triazacyclononane-N,N′,N″-triacetic acid (NOTA), 1,4,7,10-tetraazacyclododecane-1,4,7,10-tetraacetic acid (DOTA), diethylenetriaminepentaacetic acid (DTPA), 1,4,8,11-tetraazacyclotetradecane-N,N′,N″,N″-tetraacetic acid (TETA) and 1,4,7-triazacyclononane phosphinic acid (TRAP).

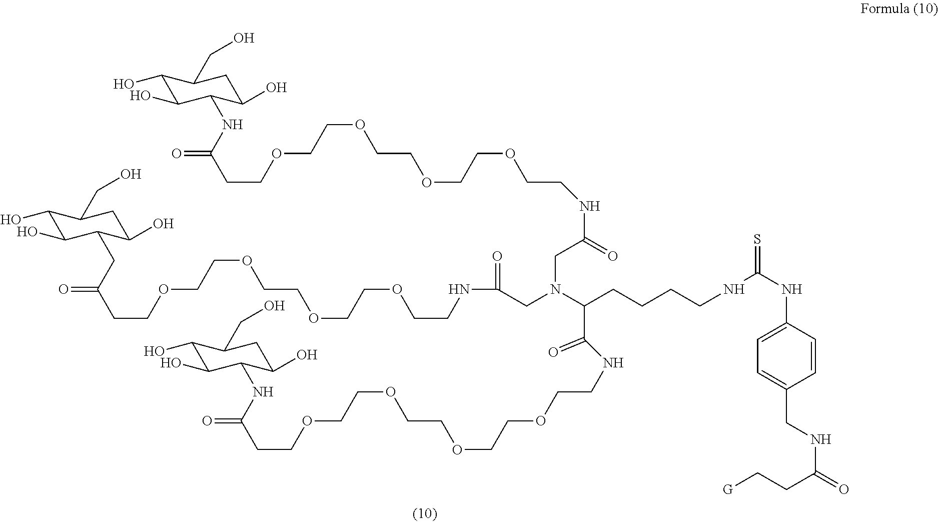

In some embodiments of the present disclosure, the chelating group G is 1,4,7-triazacyclononane-N,N′,N″-triacetic acid (NOTA), and the multivalent glyco-complex has a molecular structure as Formula (3).

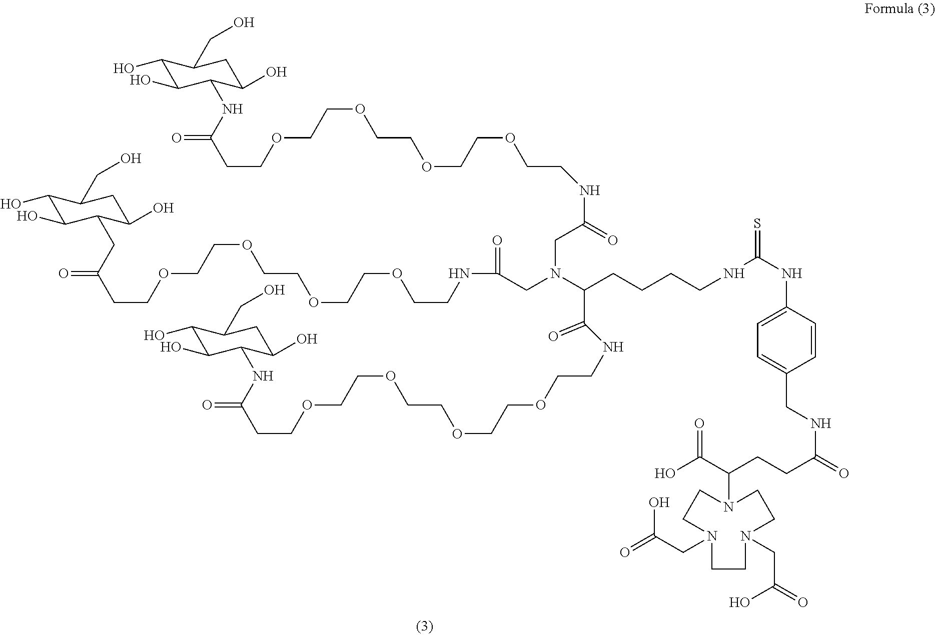 [SMILES: O=C(O)CN1CCN(CC(=O)O)CCN(C(CCC(=O)NCc2ccc(NC(=S)NCCCCC(C(=O)NCCOCCOCCOCCOCCC(=O)NC3C(O)CC(CO)C(O)C3O)N(CC(=O)NCCOCCOCCOCCOCCC(=O)CC3C(O)CC(CO)C(O)C3O)CC(=O)NCCOCCOCCOCCOCCC(=O)NC3C(O)CC(CO)C(O)C3O)cc2)C(=O)O)CC1]

In any of the abovementioned embodiments of the present disclosure, the multivalent glyco-complex further includes a radioactive nuclide complexing with the chelating group G to radiolabel the multivalent glyco-complex. In some embodiments of the present disclosure, the radioactive nuclide is Rhenium-188 (Re-188), Technetium-99 (Tc-99), Indium-111 (In-111), Lutetium-177 (Lu-177), Gallium-68 (Ga-68), Yttrium-90 (Y-90), Fluorine-18 (F-18) or Copper-64 (Cu-64).

In some embodiments of the present disclosure, the chelating group G of the multivalent glyco-complex is 1,4,7-triazacyclononane-N,N′,N″-triacetic acid (NOTA), and the radioactive nuclide is Gallium-68 (Ga-68), wherein the multivalent glyco-complex with radiolabeling having a molecular structure as Formula (4).

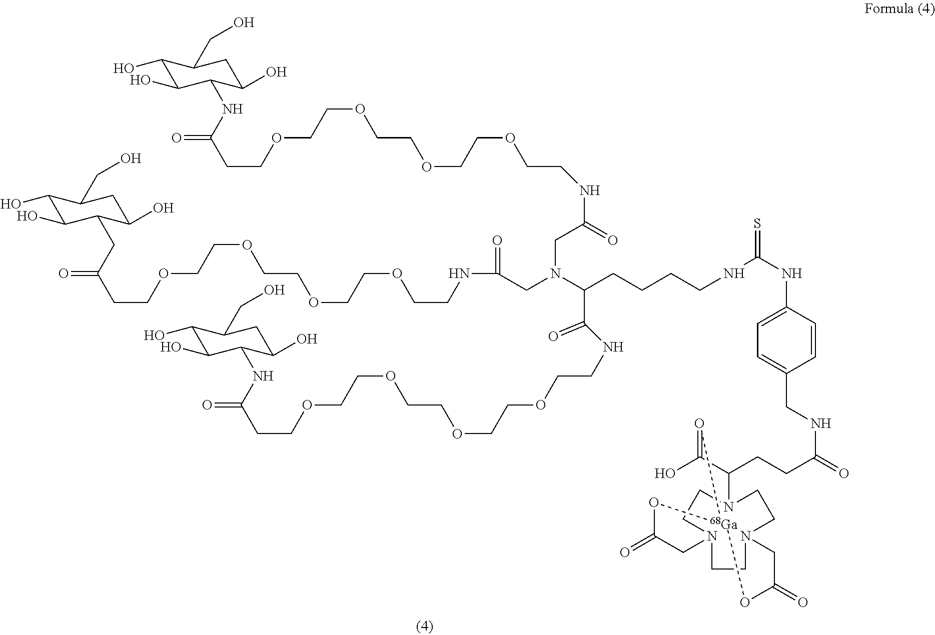

Another aspect of the present disclosure relates to an imaging agent, comprising the multivalent glyco-complex shown in any of the foregoing embodiments; and a contrast excipient.

Still another aspect of the present disclosure relates to a method of using the multivalent glyco-complex shown in any of the forgoing embodiments to diagnose a cancer.

In any optional embodiment of the instant disclosure, the cancer is selected from a group consisting of: lymphoma, multiple myeloma, testicular cancer, thyroid cancer, prostate cancer, throat cancer, cervical cancer, nasopharynx cancer, breast cancer, colorectal cancer, pancreatic cancer, gastric cancer, head-and-neck cancer, esophageal cancer, rectal cancer, bladder cancer, kidney cancer, lung cancer, liver cancer, brain cancer, melanoma and skin cancer.

Those of ordinary skill in the art the invention belongs to can fully learn the central concept, the techniques used and various embodiments of the disclosure with reference to the detailed description as follows.

### BRIEF DESCRIPTION OF THE DRAWINGS

To make the above and other objects, features, advantages and embodiments of this application more apparent and understood, the drawings are described as follows:

FIG. 1 is a representative molecular structure of the multivalent glyco-complex of the present disclosure. The multivalent glyco-complex has a molecular structure as Formula (10).

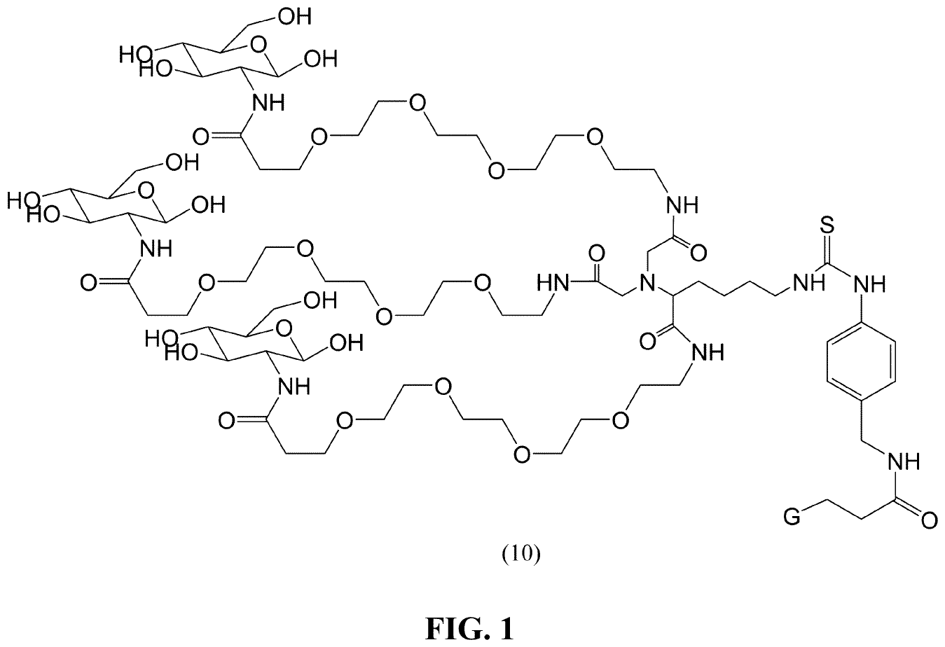

FIG. 2 is a flowchart of synthesizing a compound of Formula (1) which is a precursor used in synthesizing the multivalent glyco-complex of the present disclosure.

FIG. 3 is a mass spectrum of a compound of Formula (1).

FIG. 4 is a flowchart of synthesizing a compound of Formula (2) which is another precursor used in synthesizing the multivalent glyco-complex of the present disclosure.

FIG. 5 is a flowchart of synthesizing the multivalent glyco-complex according to an embodiment of the present disclosure. The multivalent glyco-complex has a molecular structure as Formula (3).

FIG. 6 is a mass spectrum of the multivalent glyco-complex according to an embodiment of the present disclosure. The multivalent glyco-complex has a molecular structure as Formula (3).

FIG. 7 is a flowchart of radiolabeling the multivalent glyco-complex of Formula (3) of an embodiment of the present disclosure with Gallium-68 (Ga-68) to become the multivalent glyco-complex of Formula (4).

FIG. 8 is the result of the radiochemical purity (RCP) test of the multivalent glyco-complex of Formula (4) using radio-thin layer chromatography (radio-TLC).

FIG. 9 is an embodiment of the present disclosure, which is a NanoPET/CT imaging result using the multivalent glyco-complex of Formula (4) in a breast cancer animal model.

### DETAILED DESCRIPTION OF DISCLOSED EMBODIMENTS

To make the description of the present disclosure more detailed and complete, the following illustrative written description of the examples and embodiments of this application are set forth below, but the examples and embodiments of this application are not limited thereto.

Unless otherwise stated, the scientific and technical terms used in this specification have the same meaning as commonly understood by those of ordinary skill in the art. In addition, nouns used in this specification include the singular and plural forms of the nouns, unless otherwise specified.

The word “individual” or “patient” refers to an animal capable of receiving a multivalent glyco-complex of the present disclosure. In a preferable embodiment, the animal is a mammal, and in particular, is a human.

The “cancer” stated in this specification may be a non-solid tumor or a solid tumor. For example, the cancer includes, but is not limited to tumors, such as lymphoma, multiple myeloma, testicular cancer, thyroid cancer, prostate cancer, throat cancer, cervical cancer, nasopharynx cancer, breast cancer, colorectal cancer, pancreatic cancer, gastric cancer, head-and-neck cancer, esophageal cancer, rectal cancer, bladder cancer, kidney cancer, lung cancer, liver cancer, brain cancer, melanoma and skin cancer.

As used in this specification, the term “about” generally means that an actual value is within plus or minus 10%, 5%, 1%, or 0.5% of a particular value or range. The term “about” is used herein to mean that the actual value falls within an acceptable standard error scope of the mean, as determined by those of ordinary skill in the art. It should be understood that the scopes, quantities, numerical values, and percentages used herein are modified by the term “about” with the exception of experimental examples, or unless otherwise specified. Therefore, unless otherwise stated, the numerical values or parameters disclosed in the specification and the appended claims are all approximate values and can be changed according to demand.

To resolve the problems existing in the related art, the present disclosure provides a cancer diagnostic drug with a better discrimination, especially an imaging agent used in the cancer diagnosis.

A number of examples are provided below to illustrate various implementation aspects of the present disclosure, so that those with ordinary knowledge in the technical field of the present disclosure can implement the technical content disclosed in the present invention according to the disclosure of the specification. Therefore, the examples disclosed below should not be used to limit the scope of the claims of the present disclosure. In addition, all documents cited in this specification are deemed to be fully cited and become part of this specification.

The present disclosure provides a multivalent glyco-complex containing a molecular structure as Formula (10).

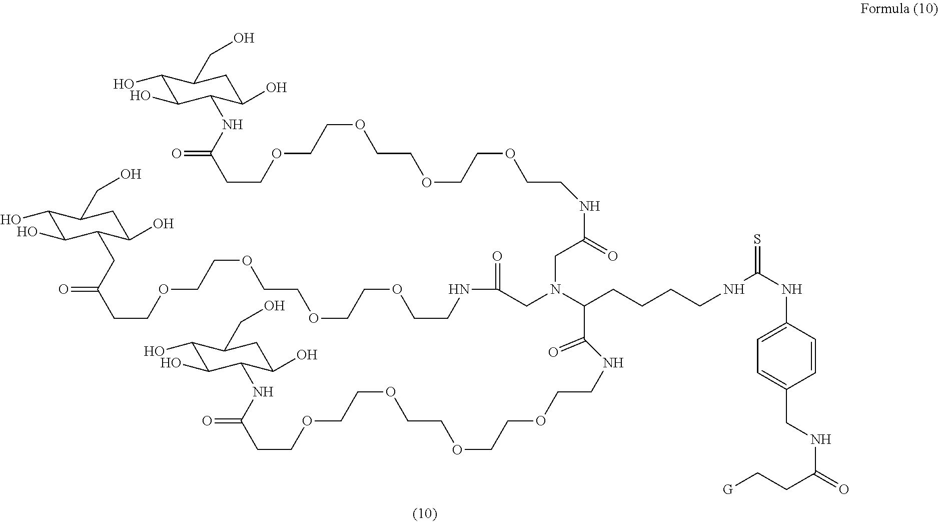

The present disclosure provides a multivalent glyco-complex of Formula (10) containing three glucose molecules connecting individually to a central nitrogen atom with a linker, and a chelating group G; wherein the linker contains at least a polyethylene glycol (PEG) molecule, and the chelating group G connects to the linker at one end closed to the central nitrogen atom in the form of p-NCS-benzyl-(chelating group G). The detailed synthesis method of the multivalent glyco-complex of Formula (10) is illustrated in the following examples.

### Example 1 Synthesizing the Multivalent Glyco-Complex of Formula (3) of the Present Disclosure

### 1.1 Preparation of a Compound of Formula (1) (NH₂-PEG-GLN)

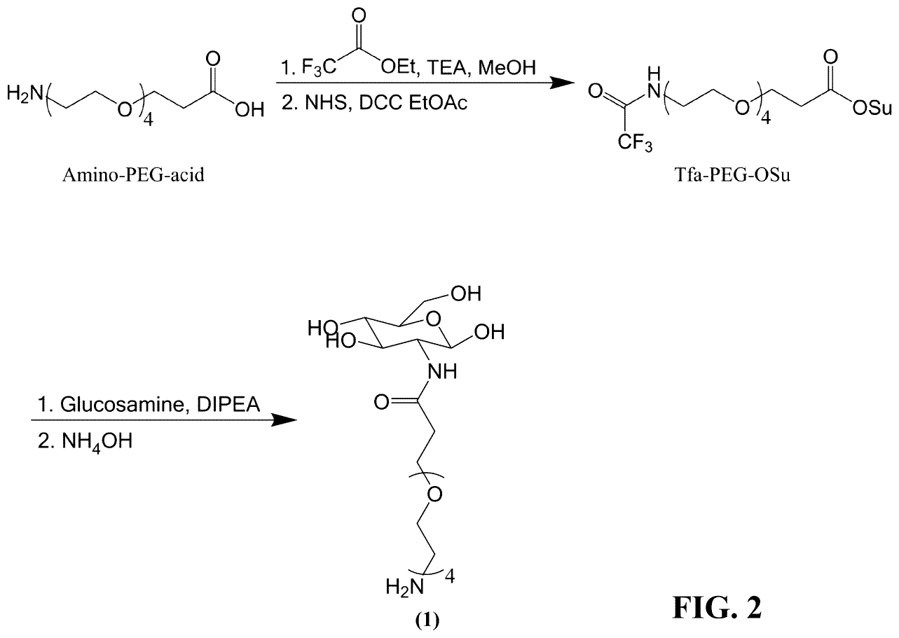

Please refer to FIG. 2 which is a flowchart of synthesizing a compound of Formula (1) (NH₂-PEG-GLN), a precursor used in synthesizing the multivalent glyco-complex of the present disclosure, wherein the PEG is a linker containing a polyethylene glycol (PEG), wherein the polyethylene glycol contains at least four ethylene glycol monomers. Besides, the GLN shown herein represents a glucose molecule.

First, as illustrated in FIG. 2, dissolve amino-PEG-acid (650 mg, Sigma-Aldrich), ethyl trifluoroacetate (2 ml, Sigma-Aldrich) and triethylamine (2 ml, Sigma-Aldrich) in 20 ml methanol (Sigma-Aldrich), and then agitate the mixed solution to allow the reaction to proceed for a period of 24 hours at ambient temperature. After then, remove the solvent by vacuum concentrator, and followed by applying Reverse-Phase HPLC to isolate the mixture. The elution gradient used is 10%-100% methanol, and the desired product is elucidated out at the elution gradient of ˜40%. After collecting the target peak, the vacuum concentrator is used to remove the solvent to obtain the intermediate product Tfa-PEG-COOH appearing as a light yellow oil form of about 210 mg with a yield of about 50%.

Secondly, dissolve Tfa-PEG-COOH (300 mg or 0.83 mmole) and N-Hydroxysuccinimide (NHS, 110 mg or 0.9 mmole, Sigma-Aldrich) in 10 ml ethyl acetate, and then add N,N′-Dicyclohexylcarbodiimide (DCC, 200 mg or 0.9 mmole, Sigma-Aldrich) into the mixture. Agitate the mixed solution to allow the reaction to proceed for a period of 24 hours at ambient temperature. Subsequently, filtrate the mixed solution to eliminate any undissolved particle(s). Then, employ a vacuum concentrator to remove the solvent until the remaining volume of the solution is less than 0.5 ml, and then dry the solution under vacuum system to obtain another intermediate product Tfa-PEG-Osu, whose molecular structure is shown in FIG. 2.

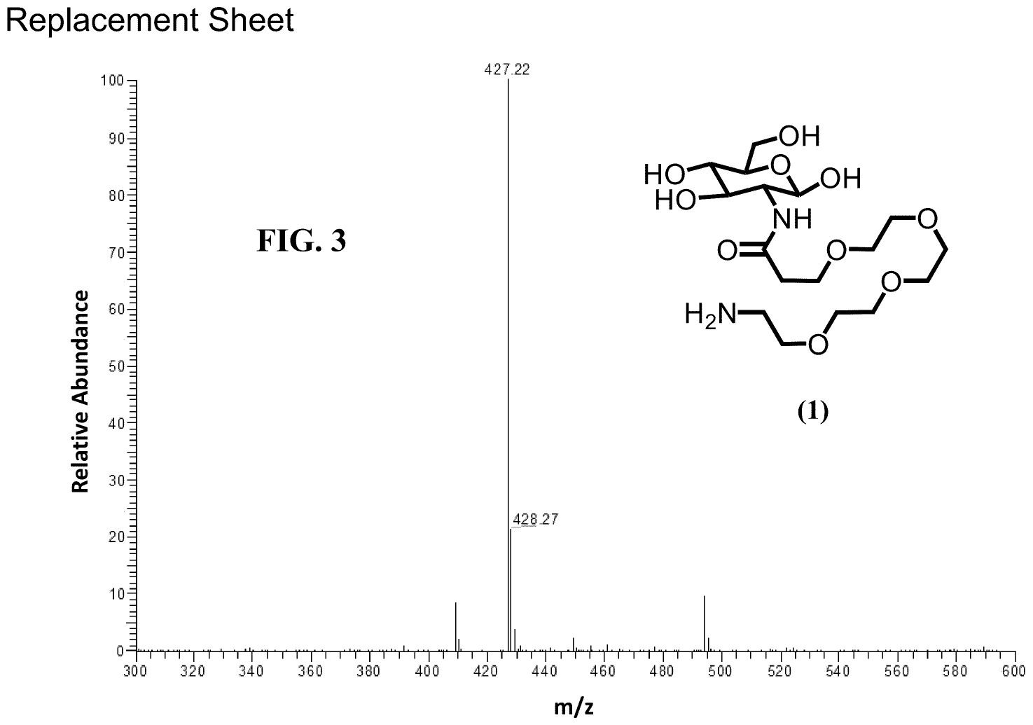

Then, dissolve Tfa-PEG-Osu (0.83 mmole), Glucosamine (180 mg or 0.83 mmole, Sigma-Aldrich) and N,N-Diisopropylethylamine (DIPEA, 1 ml, Sigma-Aldrich) in 5 ml Dimethylformamide (DMF, Sigma-Aldrich), and agitate the mixed solution to allow the reaction to proceed for a period of 24 hours at ambient temperature. After then, remove the solvent by vacuum concentrator, and followed by applying Reverse-Phase HPLC to isolate the mixture. The elution gradient used is 10%-100% methanol, and the elution peak is collected to obtain another intermediate product Tfa-PEG-GLN. Then, the deprotection reaction is carried out to remove the Tfa protecting group by hydrolyzing Tfa-PEG-GLN in an ammonia solution (pH=11.3), with agitation at ambient temperature for 48 hours. Then, remove the solvent by vacuum concentrator, followed by applying Reverse-Phase HPLC to isolate the mixture. The elution gradient used is 10%-100% methanol. The obtained product is then analyzed by using mass spectroscopy (please refer to FIG. 3) and is confirmed to be the compound of Formula (1) (NH₂-PEG-GLN) with a weight of about 218 mg.

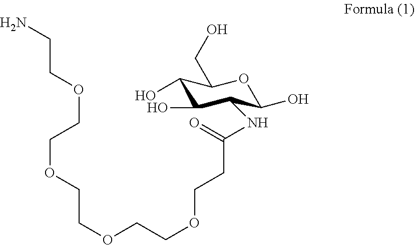

1.2 Preparation of a Compound of Formula (2) (NH₂-NTA-(PEG-GLN)₃)

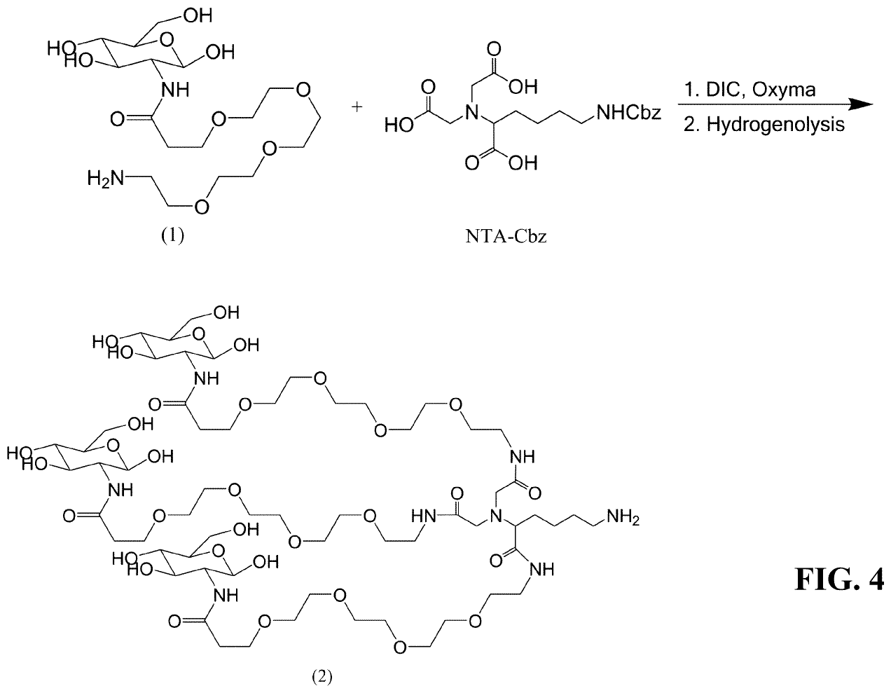

Please refer to FIG. 4, which is a flowchart of synthesizing a compound of Formula (2) (NH₂-NTA-(PEG-GLN)₃), another precursor used in synthesizing the multivalent glyco-complex of the present disclosure. The molecular structure is also shown in FIG. 4.

The synthesis method of the compound of Formula (2) is as illustrated in FIG. 4. Briefly, add N,N′-Diisopropylcarbodiimide (DIC, Sigma-Aldrich), oxyma (Sigma-Aldrich) and NTA-Cbz into the compound of Formula (1) (NH₂-PEG-GLN) to undergo the amidation reaction at ambient temperature for a period of 16 hours, and the compound of Formula (2) (NH₂-NTA-(PEG-GLN)₃) is then obtained.

### 1.3 Preparation of the Multivalent Glyco-Complex of Formula (3) (NOTA-(PEG-GLN)₃) of the Present Disclosure

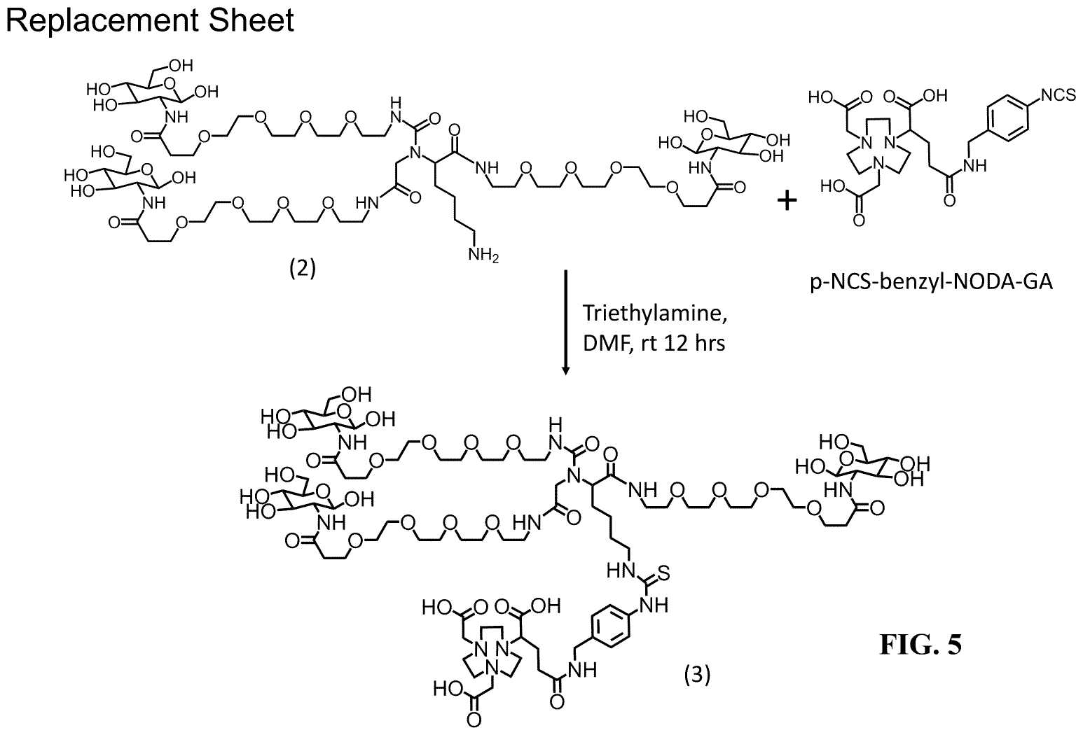

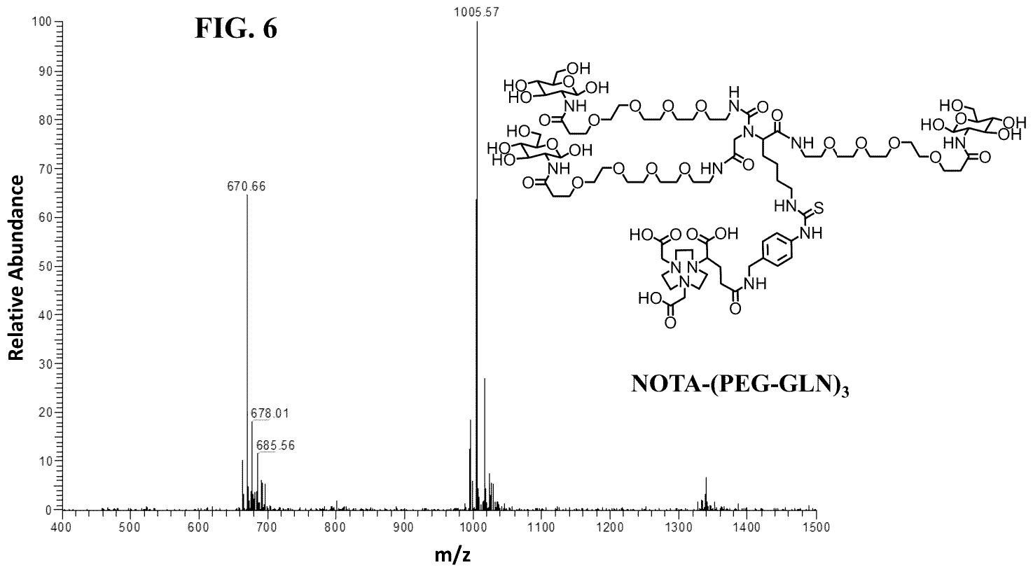

Please refer to FIG. 5, which is a flowchart of synthesizing the multivalent glyco-complex (NOTA-(PEG-GLN)₃) of Formula (3) provided in one embodiment of the present disclosure. As can be seen, the triethylamine (Sigma-Aldrich), dimethylformamide (DMF, Sigma-Aldrich), and p-NCS-benzyl-NODA-GA (Chematech) are added to the compound of Formula (2) (NH₂-NTA-(PEG-GLN)₃) to allow the amidation reaction to proceed. After reacting at ambient temperature for 12 hours, the multivalent glyco-complex of Formula (3) (NOTA-(PEG-GLN)₃) can be obtained. The obtained multivalent glyco-complex of Formula (3) is then confirmed by using mass spectrometry (Please refer to FIG. 6).

### Example 2 Accessing the Efficacy of the Radiolabeled Multivalent Glyco-Complex of the Present Disclosure by Using Lung Cancer Animal Model

2.1 Preparation of the Multivalent Glyco-Complex ⁶⁸Ga-NOTA-(PEG-GLN)₃

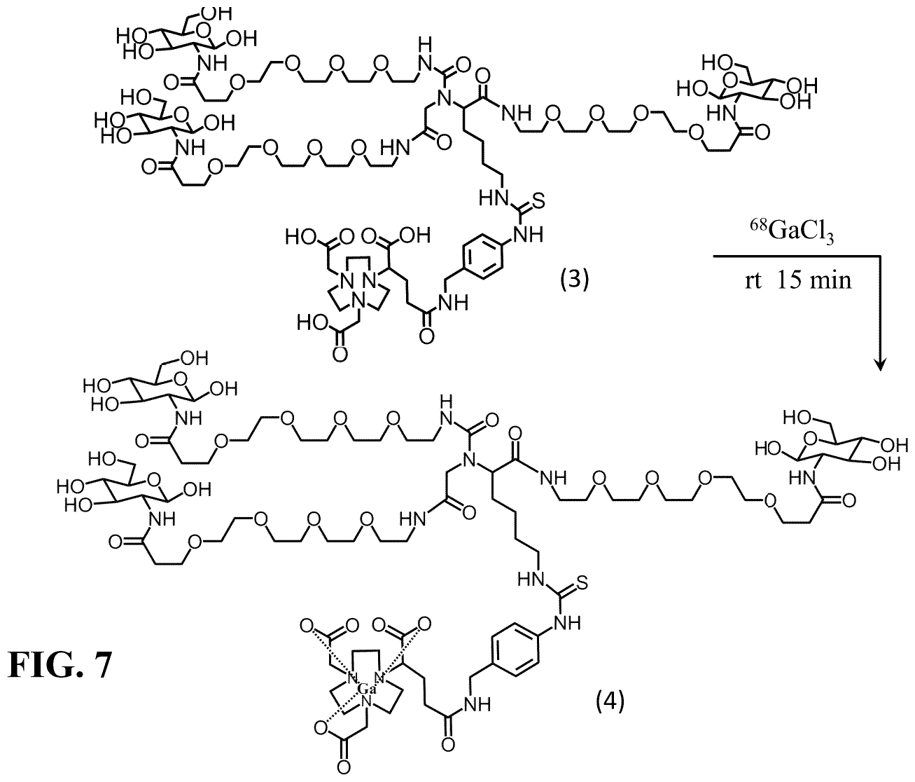

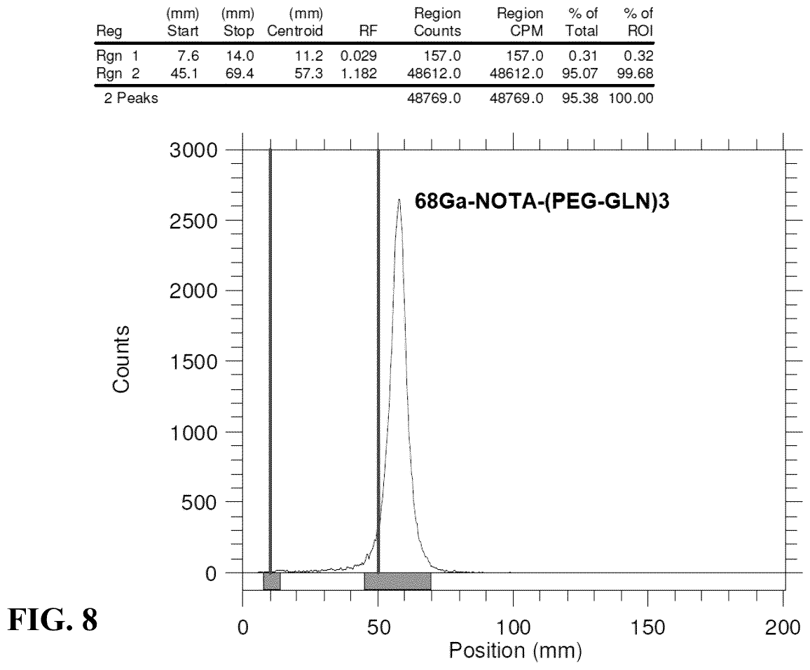

Please refer to FIG. 7, which is a flowchart of radiolabeling the multivalent glyco-complex of Formula (3) (NOTA-(PEG-GLN)₃) of an embodiment of the present disclosure with Gallium-68 (Ga-68) to become the multivalent glyco-complex of Formula (4) (⁶⁸Ga-NOTA-(PEG-GLN)₃). Firstly, use 0.1N HCl to elute the ⁶⁸Ge/⁶⁸Ga generator to obtain a solution of ⁶⁸GaCl₃. Then, add 0.5 ml ⁶⁸Ga (˜185 MBq) and 0.15 ml 1M HEPES buffer to 1 μg of the multivalent glyco-complex of Formula (3) (NOTA-(PEG-GLN)₃), and keep the mixture at ambient temperature to allow the reaction to proceed for a period of 15 minutes to obtain the radiolabeled multivalent glyco-complex of Formula (4) (⁶⁸Ga-NOTA-(PEG-GLN)₃). Lastly, the radiochemical purity (RCP) of the obtained ⁶⁸Ga-NOTA-(PEG-GLN)₃ is then confirmed to be higher than 95% by using the radio-Thin Layer Chromatography (radio-TLC) analysis, and the result is as shown in FIG. 8.

2.2 NanoPET/CT Imaging Using the Multivalent Glyco-Complex of Formula (4) (⁶⁸Ga-NOTA-(PEG-GLN)₃) as Imaging Agent in a Lung Cancer-Bearing Animal Model

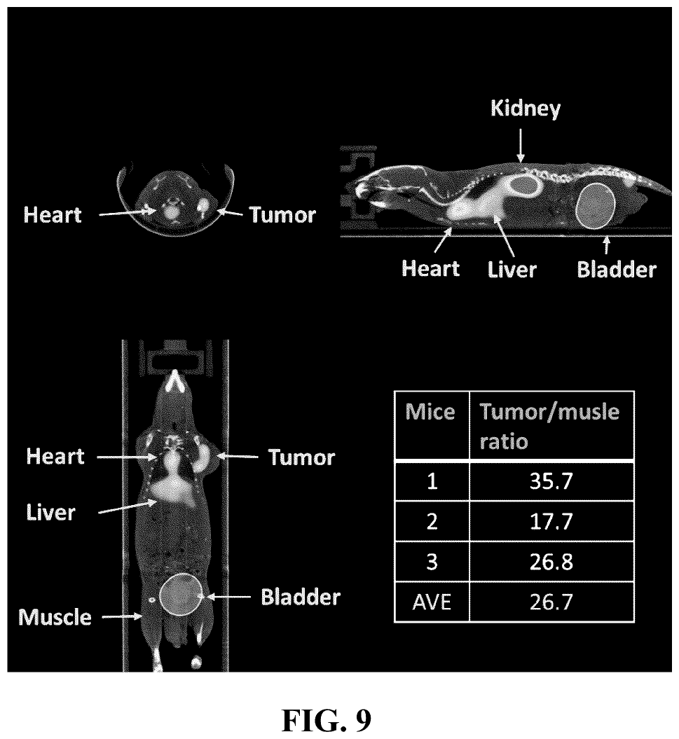

The multivalent glyco-complex (⁶⁸Ga-NOTA-(PEG-GLN)₃) of Formula (4) is firstly dissolved in 0.1 mL normal saline solution at 11.1 MBq, and then the solution is injected intravenously into nude mice bearing with NCI-H292 human lung cancer cells through the tail vein. Subsequently, nanoPET/CT imaging is performed under anesthesia with 1.5% isoflurane. After 2 hours of dynamic imaging, the result is as shown in FIG. 9. As can be seen, the result indicates that the multivalent glyco-complex of Formula (4) (⁶⁸Ga-NOTA-(PEG-GLN)₃) shows significant accumulation at the tumor sites in the animal model. The radioactivity ratio of tumor to muscle (tumor/muscle) is 27, indicating that lung cancer cells can effectively uptake the multivalent glyco-complex of Formula (4), ⁶⁸Ga-NOTA-(PEG-GLN)₃. Particularly, the uptake of this multivalent glyco-complex in the brain is extremely low in comparing with other tumor-bearing organs (Please refer to the image of FIG. 9). The result clearly indicates that the multivalent glyco-complex of Formula (4) of the present disclosure can specifically accumulate in the tumor site(s), effectively increase the uptake of the imaging agent by the tumor, and thus significantly reduce the radiation absorption dose of the brain.

According to the foregoing examples, it is obvious that the multivalent glyco-complex of Formula (10) of the present disclosure possesses the following advantages:
1. Taking advantage of the high glucose usage characteristic of most malignant tumors, the multivalent glyco-complex of the present disclosure, containing a chelating group G, a linker, and a plurality of glucose molecules in the molecular structure, can be beneficially used as imaging agent, and thus, can be efficiently taken up into tumor cells. This results in significantly improvement of the image contrast between the tumor and surrounding normal tissue(s), as well as the detection efficiency. In addition to the use in the detection of malignant tumor(s), the imaging agent containing the multivalent glyco-complex of the present disclosure can also be used in assessing of cancer therapeutic efficacy. It provides a non-invasive method to assess the appropriateness of the treatment, and if the treatment efficacy is found to be poor or inappropriate, the treatment or medication can be changed timely to avoid delaying in the therapy.
2. The multivalent glyco-complex of the present disclosure used as imaging agent is different from ¹⁸F-FDG, the most commonly used imaging agent in clinical practice nowadays. The multivalent glyco-complex molecule is significantly larger than ¹⁸F-FDG, so, the uptake in the normal brain and heart is significantly low in comparing with other tumor-bearing organs and therefore results in lower background signal in the brain and lungs. This results in significantly improvement of the image contrast between the tumor and surrounding normal tissue(s), as well as the detection efficiency.
3. The imaging agent of the multivalent glyco-complex of the present disclosure can be used for Positron Emission Tomography (PET) imaging which possesses the characteristics of non-invasiveness, high sensitivity and high image resolution.
4. When the imaging agent of the multivalent glyco-complex disclosed herein is used as positron-emitting isotope Gallium-68 (Ga-68), the radiation source can be obtained from a generator. Therefore, it is convenient for clinical use and drug preparation, without the need for a cyclotron.
5. The imaging agent of the multivalent glyco-complex of the present disclosure can be prepared in a freeze-drying kit form. After elution from the Gallium-68 generator, Gallium-68 can directly and quickly be labeled on the multivalent glyco-complex at ambient temperature. There is no need for further purification process and is convenient for clinical use. The drug cost can therefore be reduced, and the radiation absorption in the operator can also be reduced.
6. According to the foregoing examples, it is evident that the imaging agent containing the multivalent glyco-complex of Formula (4), ⁶⁸Ga-NOTA-(PEG-GLN)₃, of the present disclosure shows significant accumulation in the tumor sites in the experiment animal model. The radioactivity ratio of tumor to muscle (tumor/muscle) is 27. On the other hand, the uptake of this multivalent glyco-complex in the brain is extremely low in comparing with other tumor-bearing organs. The result clearly indicates that the multivalent glyco-complex of the present disclosure can specifically accumulate in the tumor site(s), effectively increase the uptake of the imaging agent by the tumor, and thus significantly reduce the radiation absorption of the brain.

Although this application has been disclosed above by way of embodiments, it is not intended to limit this application. Various changes and modifications can be made by those of ordinary skill in the art without departing from the spirit and scope of this application. Therefore, the scope of protection of this application is defined by the appended claims.

[Symbol description] None

What is claimed is:

1. A multivalent glyco-complex, comprising the structure of Formula (10): 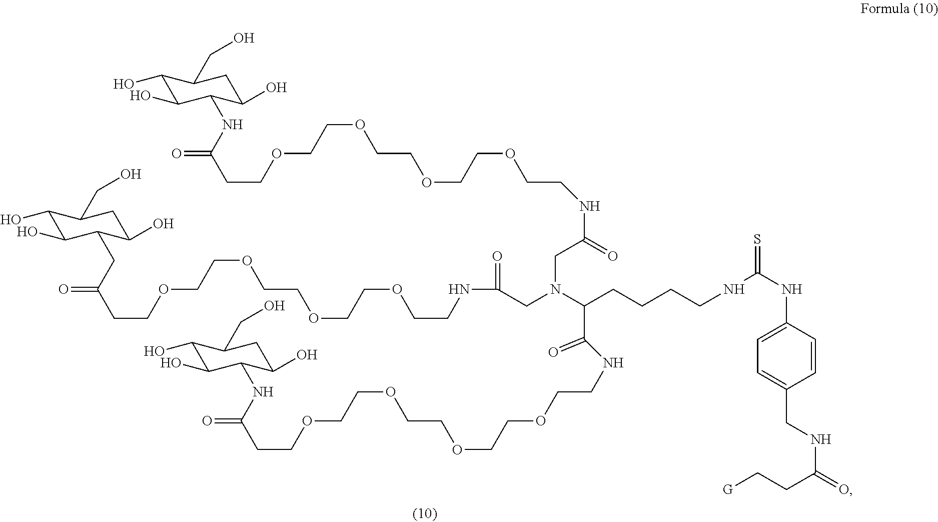
wherein G is a chelating group and is selected from a group consisting of: 1,4,7-triazacyclononane-N,N′,N″-triacetic acid (NOTA), 1,4,7,10-tetraazacyclododecane-1,4,7,10-tetraacetic acid (DOTA), diethylenetriaminepentaacetic acid (DTPA), 1,4,8,11-tetraazacyclotetradecane-N,N′,N″,N′-tetraacetic acid (TETA) and 1,4,7-triazacyclononane phosphinic acid (TRAP).

2. The multivalent glyco-complex according to claim 1, wherein the chelating group G is 1,4,7-triazacyclononane-N,N′,N″-triacetic acid (NOTA), and the multivalent glyco-complex comprising the structure of Formula (3):

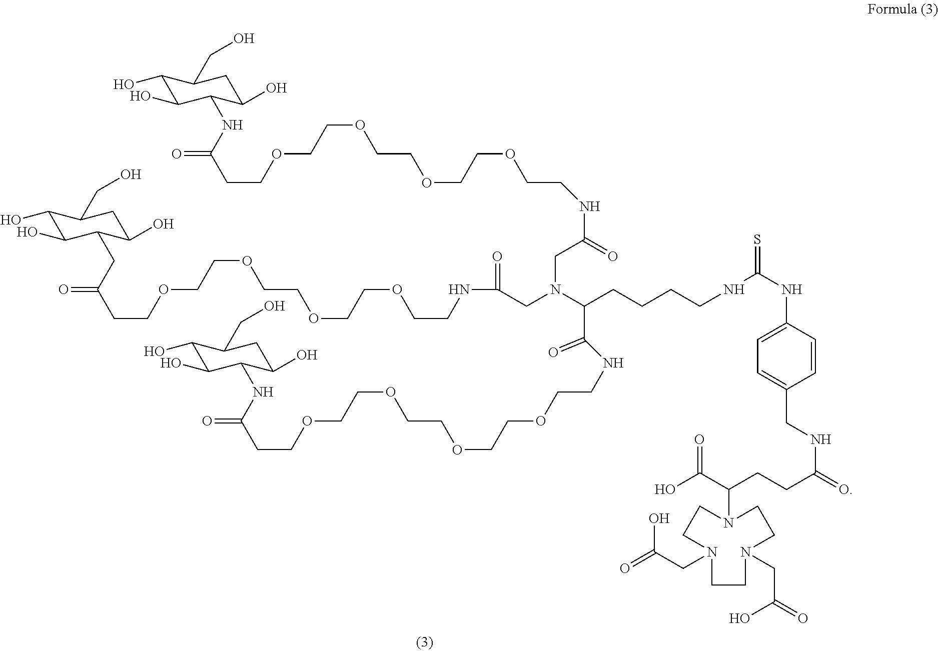 [SMILES: O=C(O)CN1CCN(CC(=O)O)CCN(C(CCC(=O)NCc2ccc(NC(=S)NCCCCC(C(=O)NCCOCCOCCOCCOCCC(=O)NC3C(O)CC(CO)C(O)C3O)N(CC(=O)NCCOCCOCCOCCOCCC(=O)CC3C(O)CC(CO)C(O)C3O)CC(=O)NCCOCCOCCOCCOCCC(=O)NC3C(O)CC(CO)C(O)C3O)cc2)C(=O)O)CC1]

3. The multivalent glyco-complex according to claim 1, further comprising a radioactive nuclide complexing with the chelating group G to radiolabel the multivalent glyco-complex, wherein the radioactive nuclide is Rhenium-188 (Re-188), Technetium-99 (Tc-99), Indium-111 (In-111), Lutetium-177 (Lu-177), Gallium-68 (Ga-68), Yttrium-90 (Y-90), Fluorine-18 (F-18) or Copper-64 (Cu-64).

4. The multivalent glyco-complex according to claim 3, wherein the radioactive nuclide is gallium-68 (Ga-68).

5. The multivalent glyco-complex according to claim 3, wherein the chelating group G is 1,4,7-triazacyclononane-N,N′,N″-triacetic acid (NOTA), and the radioactive nuclide is gallium-68 (Ga-68).

6. An imaging agent, comprising:
the multivalent glyco-complex according to claim 1; and
a contrast excipient.

7. A method of using the multivalent glyco-complex according to claim 1 to diagnose a cancer.

8. The method according to claim 7, wherein the cancer is selected from a group consisting of: lymphoma, multiple myeloma, testicular cancer, thyroid cancer, prostate cancer, throat cancer, cervical cancer, nasopharynx cancer, breast cancer, colorectal cancer, pancreatic cancer, gastric cancer, head-and-neck cancer, esophageal cancer, rectal cancer, bladder cancer, kidney cancer, lung cancer, liver cancer, brain cancer, melanoma and skin cancer.
